(19) United States
(12) Patent Application Publication
(10) Pub. No.: US 2021/0386353 A1
(43) Pub. Date: Dec. 16, 2021

(54) AMBULATORY HEART MONITOR WITH CONDUCTIVE ADHESIVE CONNECTION TO ELECTRONICS MODULE

(71) Applicant: Cardiac Insight, Inc., Bellevue, WA (US)

(72) Inventors: David Hadley, Woodinville, WA (US); Robert ODell, Newcastle, WA (US)

(21) Appl. No.: 17/154,245

(22) Filed: Jan. 21, 2021

Related U.S. Application Data
(63) Continuation of application No. 15/666,016, filed on Aug. 1, 2017, now abandoned.
(60) Provisional application No. 62/371,112, filed on Aug. 4, 2016.

(51) Int. Cl. A61B 5/335; A61B 5/00; A61B 5/259

(52) U.S. Cl. CPC A61B 5/335; A61B 5/6833; A61B 5/01; A61B 5/259; A61B 5/68335

(57) ABSTRACT

An ambulatory monitor includes a conductive adhesive for electrically coupling a data recording module with a patient adhereable flexible patch. The ambulatory monitor includes a skin adhereable flexible patch and a data recording module that can be mounted to and demounted from the patch.

### CROSS-REFERENCE TO RELATED APPLICATION(S)

This application is a continuation of U.S. patent application Ser. No. 15/666,016, filed Aug. 1, 2017, which claims the benefit of U.S. Patent Application No. 62/371,112, filed Aug. 4, 2016, the contents of which are hereby incorporated by reference in their entireties.

### BACKGROUND

Electrocardiography (ECG or EKG) is often used to assess the electrical and muscular functions of the heart using electrodes placed on the skin. The electrodes detect electrical variations that arise from heart muscle depolarization and repolarization during each heartbeat. EGG is a commonly employed method to assess the health of a person's heart.

In many instances, ECG is performed in a clinical setting, such as in a hospital or in a doctor's office. Performing ECG in a clinical setting, however, tends to discourage widespread application of ECG as a precautionary method for the detection of latent, undetected heart conditions, such as congenital heart defects. Moreover, time constraints in the clinical setting tend to limit the timespan over which the ECG is performed, which may inhibit detection of intermittently observable heart conditions.

Ambulatory physiological data monitors can be used to gather physiological data outside the clinical setting, thereby potentially enabling wider application of patient diagnoses than offered in the clinical setting. Ambulatory physiological data monitors also offer the potential of gathering physiological data over extended timespans that may not be practical in clinical settings. Gathering physiological data over extended timespans, however, can be challenging in practice in view of environmental factors such as perspiration, water ingress during bathing, environmental exposure to dirt and/or moisture, etc. Such environmental factors can degrade communication of an electrical signal from the person's skin to a data recording module. Moreover, such environmental factors can result in contamination of the ambulatory physiological data monitor over time. Accordingly, improved ambulatory physiological data monitors that can be used to record physiological data over extended periods of time in view of the applicable environment are of interest.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Improved ambulatory monitors for recording physiological data include a detachable physiological data recording module that includes electrical contacts that are interfaced with corresponding electrical contacts of a patient adhereable patch via a conductive adhesive. The conductive adhesive prevents inadvertent separation of the data recording electrical contacts from the corresponding patient adhereable patch electrical contacts and enhances the electrical connection. In many embodiments, the physiological data recording module can be demounted by hand (e.g., by grasping the physiological data recording module and rocking the module relative to the patch to sequentially peel the module electrical contacts from the patch electrical contacts and the conductive adhesive). Data can also be downloaded from the data recording module.

Thus, in one aspect, an ambulatory monitor is provided. The ambulatory monitor includes a physiological data recording module and a flexible patch configured to be attached to a user's skin. The flexible patch includes: (a) a biocompatible layer configured to be coupled with the user's skin, (b) an upper layer overlying the biocompatible layer, (c) a first electrode coupled with the upper layer so as to be operatively coupled with the user's skin when the flexible patch is attached to the user's skin, (d) a patch first electrical contact electrically connected with the first electrode, (e) a conductive adhesive interfaced with the patch first electrical contact, (f) a second electrode coupled with the upper layer so as to be operatively coupled with the user's skin when the flexible patch is attached to the user's skin, (g) a patch second electrical contact electrically connected with the second electrode, and (h) a conductive adhesive interfaced with the patch second electrical contact. The physiological data recording module includes a module first electrical contact and a module second electrical contact. The physiological data recording module is configured to be mounted to the flexible patch so as to electrically couple the module first electrical contact with the patch first electrical contact and the module second electrical contact with the patch second electrical contact. The physiological data recording module is configured to store physiological data of the user generated from an electrical signal from the first and second electrodes.

In many embodiments of the ambulatory monitory, the upper layer includes an upper layer surrounding portion to which each of the patch first and second electrodes is mounted, an upper layer first tab portion to which the patch first electrical contact is mounted, and an upper layer second tab portion to which the patch second electrical contact is mounted. In many embodiments, the upper layer surrounding portion forms an aperture into which each of the upper layer first and second tab portions extends. In many embodiments, the flexible patch includes an upper layer adhesive layer configured to bond the upper layer surrounding portion to the biocompatible layer. At least a portion of each of the upper layer first and second tab portions can extend beyond the upper layer adhesive layer so that the at least a portion of each of the upper layer first and second tab portions is not bonded to the biocompatible layer.

In many embodiments of the ambulatory monitory, the upper layer first tab portion includes a first end portion, a second end portion, and a central portion disposed between the first end portion and the second end portion. In a similar manner, upper layer second tab portion can include a first end portion, a second end portion, and a central portion disposed between the first end portion and the second end portion. In many embodiments, the flexible patch includes an end portion adhesive layer coupled with each of the first and second end portions of each of the upper layer first and second tab portions to bond the data recording module to each of the first and second end portions of each of the upper layer first and second tab portions. In many embodiments, the patch first electrical contact is mounted to the central portion of the upper layer first tab portion and the patch second electrical contact is mounted to the central portion of the upper layer second tab portion. Any suitable adhesive can be used as the end portion adhesive layer, for example, a suitable non-conductive adhesive.

In many embodiments of the ambulatory monitory, the module first and second electrical contacts protrude from the module so as to be embedded into the conductive adhesive. For example, the physiological data recording module can include a housing having a lower surface beyond which each of the module first and second electrical contacts protrudes.

In many embodiments of the ambulatory monitor, the physiological data recording module is protected from moisture ingression. For example, the ambulatory monitor can include a water-resistant cover configured to be bonded to or made part of the flexible patch to cover the physiological data recording module to protect the physiological data recording module from moisture ingression.

In many embodiments, the ambulatory monitor includes formed electrical traces that connect the electrodes with the patch electrical contacts. For example, the ambulatory monitory can include a first electrical trace formed on the upper layer and electrically connecting the first electrode with the patch first electrical contact and a second electrical trace formed on the upper layer and electrically connecting the second electrode with the patch second electrical contact.

In many embodiments of the ambulatory monitor, the physiological data recording module is demountable from the flexible patch without employing tools. For example, in many embodiments, the user can demount the physiological data recording module from the flexible patch by hand.

In many embodiments of the ambulatory monitor, a conductive material is used to electrically couple the electrodes with the user's skin. For example, in many embodiments: (a) each of the first and second electrodes is formed on the upper layer, (b) the biocompatible layer has a first electrode aperture aligned with the first electrode and a second electrode aperture aligned with the second electrode, (c) the flexible patch includes a conductive material interfaced with the first electrode and extending through the first electrode aperture for electrically coupling the first electrode to the user's skin, and (d) the flexible patch includes a conductive material interfaced with the second electrode and extending through the second electrode aperture for electrically coupling the second electrode to the user's skin.

A person of skill will appreciate that the ambulatory monitor can include any suitable number of electrodes (e.g., two, three, four, five, six, or more electrodes) that are operatively coupled with the user's skin when the flexible patch is attached to the user's skin. For example, the flexible patch can further include: (a) a third electrode coupled with the upper layer so as to be operatively coupled with the user's skin when the flexible patch is attached to the user's skin; (b) a patch third electrical contact electrically connected with the third electrode; and (c) a conductive adhesive interfaced with the patch third electrical contact. The physiological data recording module can further include a module third electrical contact. The physiological data recording module can be configured to: (a) be mounted to the flexible patch so as to electrically couple the module third electrical contact with the patch third electrical contact; and (b) store physiological data of the user generated via the first, second, and third electrodes.

In another aspect, a method is provided for transferring an electrical signal from a patient to a physiological data recording module. The method includes supporting a first electrode and a second electrode in electrical connection with the patient via a flexible patch adhered to the patient. A patch first electrical contact is adhered with a module first electrical contact of the physiological data recording module via a conductive adhesive. The patch first electrical contact is electrically coupled with the first electrode. A patch second electrical contact is adhered with a module second electrical contact of the physiological data recording module via a conductive adhesive. The patch second electrical contact is electrically coupled with the second electrode. The electrical signal is transferred from the patient to the physiological data recording module via a conductive path comprising the first and second electrodes, the patch first and second electrical contacts, and the module first and second electrical contacts.

In many embodiments, the method for transferring an electrical signal from a patient to a physiological data recording module includes demounting the physiological data recording module from the flexible patch without the use of tools. For example, the method can include demounting the physiological data recording module from the flexible patch by hand. In many embodiments of the method, demounting the physiological data recording module from the flexible patch by hand includes rocking the physiological data recording module relative to the flexible patch.

Any suitable ambulatory monitory can be used to perform acts of the method for transferring an electrical signal from a patient to a physiological data recording module. For example, the flexible patch can include a biocompatible layer and an upper layer overlying the biocompatible layer. The upper layer can include an upper layer surrounding portion, an upper layer first tab portion, and an upper layer second tab portion. The upper layer surrounding portion can form an aperture into which each of the upper layer first tab portion and the upper layer second tab portion extends. The method can include: (a) adhering the upper layer surrounding portion to the biocompatible layer via an intervening adhesive layer, (b) supporting the patch first electrical contact on the upper layer first tab portion, (c) supporting the patch second electrical contact on the upper layer second tab portion, and (d) accommodating relative movement between the biocompatible layer and each of the upper layer first and second tab portions by not adhering at least a portion of each of the upper layer first and second tab portions to the biocompatible layer. The first electrode can be electrically connected to the patch first electrical contact via a first conductive trace formed on the upper layer. The second electrode can be electrically connected to the patch second electrical contact via a second conductive trace formed on the upper layer. The method can include protecting the physiological data recording module from water ingression via a water-resistant cover bonded to the flexible patch. The upper layer first tab portion can include a first end portion, a second end portion, and a central portion disposed between the first end portion and the second end portion. The upper layer second tab portion can include a first end portion, a second end portion, and a central portion disposed between the first end portion and the second end portion. The method can include: (a) adhering the physiological data recording module to each of the first and second end portions of each of the upper layer first and second tab portions using a non-conducting adhesive, (b) supporting the patch first electrical contact via the central portion of the upper layer first tab portion, and (c) supporting the patch second electrical contact via the central portion of the upper layer second tab portion. The physiological data recording module can include a housing having a lower surface beyond which each of the module first and second electrical contacts protrude.

A person of skill will appreciate that the method for transferring an electrical signal from a patient to a physiological data recording module can employ an ambulatory monitor that includes any suitable number of electrodes (e.g., two, three, four, five, six, or more electrodes) that are operatively coupled with the user's skin when the flexible patch is attached to the user's skin. For example, the method for transferring an electrical signal from a patient to a physiological data recording module can further include: (a) supporting a third electrode in electrical connection with the patient via the flexible patch; (b) adhering a patch third electrical contact with a module third electrical contact of the physiological data recording module via a conductive adhesive, the patch third electrical contact being electrically coupled with the third electrode; and (c) generating the electrical signal via the first, second, and third electrodes.

For a fuller understanding of the nature and advantages of the present invention, reference should be made to the ensuing detailed description and accompanying drawings.

### BRIEF DESCRIPTION OF THE DRAWINGS

FIG. 1 shows an ambulatory monitor, in accordance with many embodiments, adhered to the chest of a patient.

FIG. 2 shows a plan view of the ambulatory monitor of FIG. 1.

FIG. 3 shows an exploded view of the ambulatory monitor of FIG. 1.

FIG. 4 shows a plan view of a free-floating tab of a flexible patch of the ambulatory monitor of FIG. 1 for electrically coupling a data recording module of the ambulatory monitory of FIG. 1 to electrodes supported by the flexible patch, in accordance with many embodiments.

FIG. 5 shows an exploded cross-sectional view of the ambulatory monitor of FIG. 1.

FIG. 6 illustrates demounting the data recording module of the ambulatory monitor of FIG. 1 from the flexible patch of the ambulatory monitor of FIG. 1 by hand and without tools, in accordance with many embodiments.

FIG. 7 is a simplified block diagram of acts of a method for transferring an electrical signal from a patient to a physiological data recording module.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

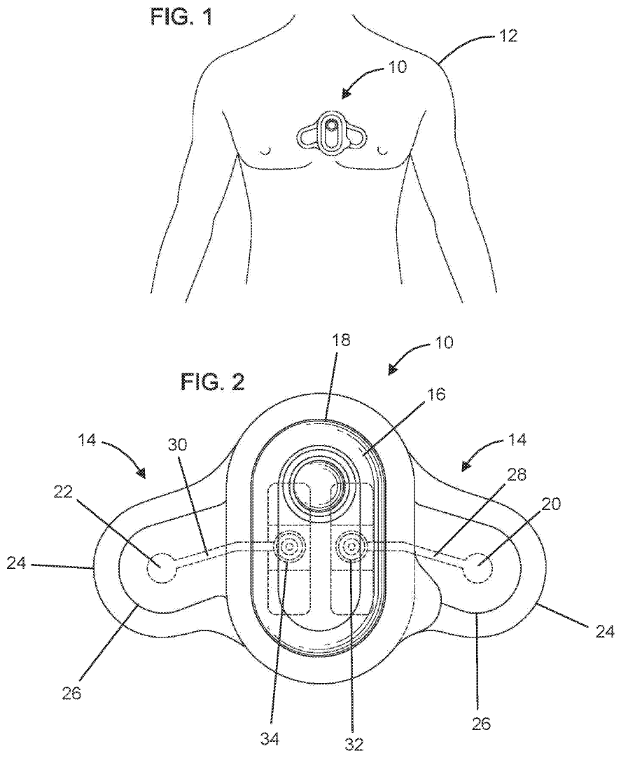

Referring now to the drawings, in which like reference numerals represent like parts throughout the several views, FIG. 1 shows an ambulatory monitor 10 adhered to the chest of a user 12. In embodiments described herein, the ambulatory monitory 10 is configured to record ECG data for the user 12 that is generated via two electrodes coupled with the user's skin. The ambulatory monitor 10 can, however, be adapted to record any suitable physiological data of the user 12. For example, the ambulatory monitor 10 can be configured to record body temperature, body motion, and/or blood oxygen saturation. A person of skill will appreciate that the ambulatory monitor 10 can be configured with any suitable number of electrodes (e.g., two, three, four, five, six, or more electrodes) that are coupled with the user's skin to record any suitable physiological data of the user 12.

FIG. 2 shows a plan view of the ambulatory monitor 10. In the illustrated embodiment, the ambulatory monitor 10 includes a flexible patch 14, a physiological data recording module 16, and a water resistant cover 18. The flexible patch 14 includes a first electrode 20, a second electrode 22, a biocompatible layer 24, an upper layer 26, a first conductive trace 28, a second conductive trace 30, a patch first electrical contact 32, and a patch second electrical contact 34. The flexible patch 14 is adhereable to the user skin and is flexible to accommodate change in shape of the user's skin to maintain coupling between the first and second electrodes 20, 22 and the user's skin and to inhibit patient discomfort. The first conductive trace 28 electrically couples the first electrode 20 with the patch first electrical contact 32. The second conductive trace 30 electrically couples the second electrode 22 with the patch second electrical contact 34. The patch first and second electrical contacts 32, 34 are configured to be electrically coupled with the data recording module 16 to transfer an electrical signal from the patient 12 to the data recording module 16 via the first and second electrodes 20, 22 and the first and second conductive traces 28, 30.

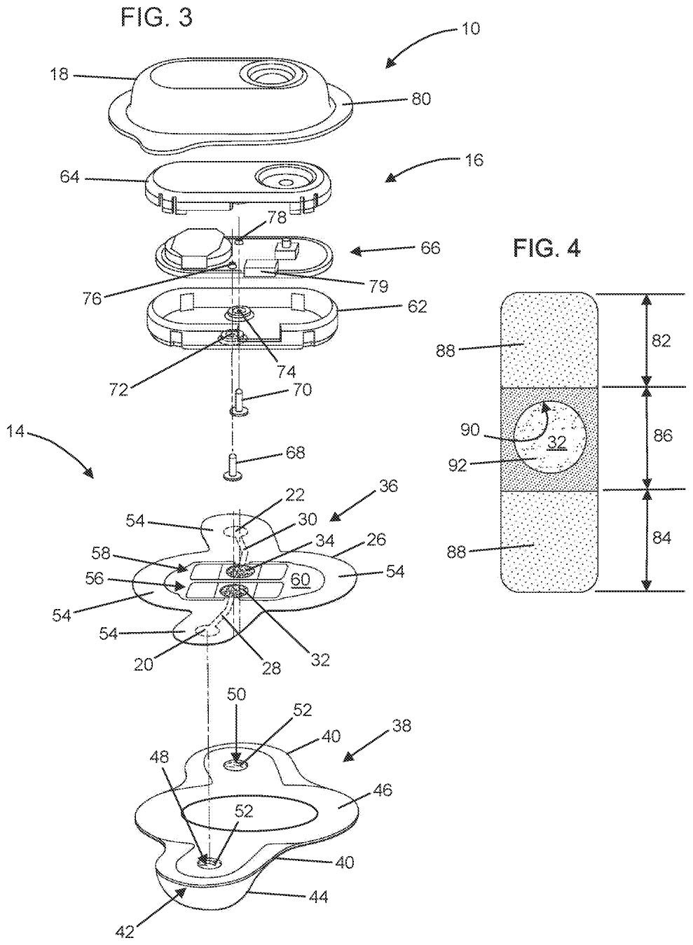

FIG. 3 shows an exploded view of the ambulatory monitor 10. The flexible patch 14 includes an upper layer assembly 36 and a biocompatible layer assembly 38. The biocompatible layer assembly 38 includes a biocompatible layer 40, a skin adhesive layer, 42, a peel ply 44, and an upper layer adhesive layer 46. The biocompatible layer 40 can be made from any suitable material and have any suitable thickness. For example, the biocompatible layer 40 can be made from a vapor permeable fabric coated with a hydrocolloid adhesive, with a thickness of less than 1 mm. The skin adhesive layer 42 can cover any suitable portion of the bottom surface of the biocompatible layer 40 and is configured to adhere the biocompatible layer 40 to the user's skin. The peel ply 44 covers the skin adhesive layer 42 to protect the skin adhesive layer 42 prior to adhering the flexible patch 14 to the user's skin. The peel ply 44 is removable to expose the skin adhesive layer 42 so that the skin adhesive layer 42 can be interfaced with the user's skin. The upper layer adhesive layer 46 is sized and shaped to adhere the biocompatible layer 40 with specific portions of the upper layer assembly 36 and to not adhere the biocompatible layer 40 with first and second tabbed portions of the upper layer assembly 36 as described herein. The biocompatible layer 40 and the upper layer adhesive layer 46 have a first electrode aperture 48 and a second electrode aperture 50 that accommodate a conductive material 52 that electrically couples the user's skin with the first and second electrodes 20, 22.

The upper layer assembly 36 includes the first and second electrodes 20, 22, the upper layer 26, the first conductive trace 28, the second conductive trace 30, the patch first electrical contact 32, and the patch second electrical contact 34. The upper layer 26 includes an upper layer surrounding portion 54 on which each of the first and second electrodes 20, 22 is formed, an upper layer first tab portion 56 on which the patch first electrical contact 32 is formed, and an upper layer second tab portion 58 on which the patch second electrical contact 34 is formed. The upper layer surrounding portion 54 forms an aperture 60 into which each of the upper layer first tab portion 56 and the upper layer second tab portion 58 extends. The first conductive trace 28 is formed on the upper layer 26 and extends to electrically couple the first electrode 20 and the patch first electrical contact 32. The second conductive trace 30 is formed on the upper layer 26 and extends to electrically couple the second electrode 22 and the patch second electrical contact 34. In the illustrated embodiment, the first and second electrodes 20, 22, the first and second conductive traces 28, 30, and the patch first and second electrical contacts 32, 34 are formed on a lower surface of the upper layer 26 so as to be disposed between the upper layer 26 and the biocompatible layer 40. As described herein, each of the upper layer first and second tab portions 56, 58 include an aperture through which the respective patch first and second electrical contact 32, 34 is electrically coupled with the data recording module 16. The upper layer adhesive layer 46 is configured to bond upper layer assembly 36 to the biocompatible layer 40 without bonding the upper layer first and second tab portions 56, 58 to the biocompatible layer 40 so as to accommodate movement of the upper layer first and second tab portions 56, 58 relative to the biocompatible layer 40.

The data recording module 16 is configured to record physiological data for the user 12 generated via an electrical signal from the patient that is transferred to the data recording module 16 via the first and second electrodes 20, 22, the first and second conductive traces 28, 30, and the patch first and second electrical contacts 32, 34. The data recording module 16 includes a lower housing 62, an upper housing 64, an electronic assembly 66, a module first electrical contact 68, and a module second electrical contact 70. The lower housing 62 forms apertures 72, 74 through which the module first and second electrical contacts 68, 70 extend. The electronic assembly 66 includes connectors 76, 78 into which module first and second electrical contacts 68, 70 extend, thereby electrically connecting the electronic assembly 66 with the module first and second electrical contacts 68, 70. The electronic assembly 66 includes an electrical connector 79 that serves as a data port for data transfer to and/or from the electronic assembly 66. The electrical connector 79 is mounted flush within an aperture formed via the lower housing 62 and the upper housing 64. The lower housing 62 forms a recess configured to accommodate and position the electronic assembly 66 so as to align the connectors 76, 78 with the apertures 72, 74. The upper housing 64 is configured to be mountable to the lower housing 62 so as to enclose the electronic assembly 66. In the illustrated embodiment, the upper housing 64 can be demounted from the lower housing 62 to provide access to the electronic assembly 66 for downloading physiological data from the electronic assembly 66 and/or to charge the electronic assembly 66. In many embodiments, the electronic assembly 66 includes one or more batteries to power the electronic assembly 66, a processor or a controller to generate physiological data from the electrical signal received from the user 12, and a tangible memory for storing the physiological data.

The water resistant cover 18 is configured to protect the data recording module 16 from water ingression. The water-resistant cover 18 includes a perimeter attachment flange 80 configured to be adhered to the upper layer 26. The cover 18 forms a recess sized to accommodate the data recording module 16, thereby enclosing the data recording module 16 between the cover 18 and the flexible patch 14.

FIG. 4 shows a plan view of the upper layer first tab portion 56. The upper layer second tab portion 58 is configured similar to the upper layer first tab portion 56. The upper layer first tab portion 56 comprises a first end portion 82, a second end portion 84, and a central portion 86 disposed between the first end portion 82 and the second end portion 84. An end portion adhesive layer 88 is adhered to each of the first and second end portions 82, 84. The central portion 86 has an aperture 90 through which the patch first electrical contact 32 is exposed. A conductive adhesive 92 is adhered to the patch first electrical contact 32. The conductive adhesive 92 is configured to be interfaced with the module first electrical contact 68 to maintain and/or enhance electrical coupling between the module first electrical contact 68 and the patch first electrical contact 32.

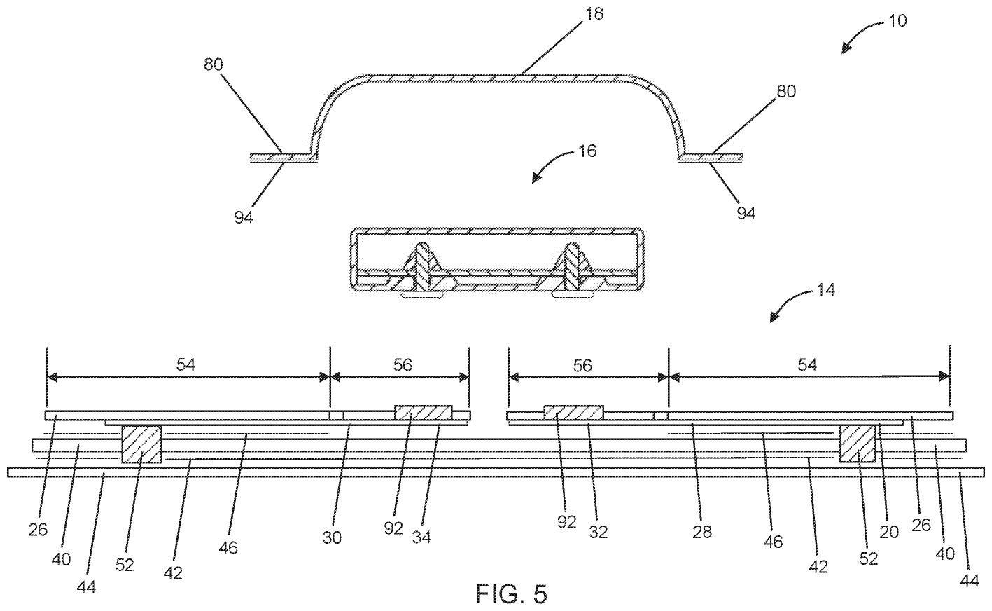

FIG. 5 shows an exploded cross-sectional view of the ambulatory monitor 10. From top to bottom, the flexible patch 14 includes the upper layer 26, the biocompatible layer 40, and the removable peel ply 44. The upper layer 26 includes the surrounding portion 54, the first tab portion 56, and the second tab portion 58. The first electrode 20, the first conductive trace 28, and the patch first electrical contact 32 can be created by forming a corresponding conductive layer on the bottom surface of the upper layer 26. In a similar way, the second electrode 22, the second conductive trace 30, and the patch second electrical contact 34 can be created by forming a corresponding conductive layer on the bottom surface of the upper layer 26. The upper layer adhesive layer 46 bonds the surrounding portion 54 of the upper layer assembly 36 to the biocompatible layer 40 without bonding the first and second tab portions 56, 58 to the biocompatible layer 40. The peel ply 44 is removable to expose the skin adhesive layer 42, which is used to adhere the flexible patch 14 to the user's skin. The conductive material 52 serves to electrically couple the first and second electrodes 20, 22 with the user's skin. The water-resistant cover 18 is adhered to the upper layer 26 via a cover adhesive layer 94.

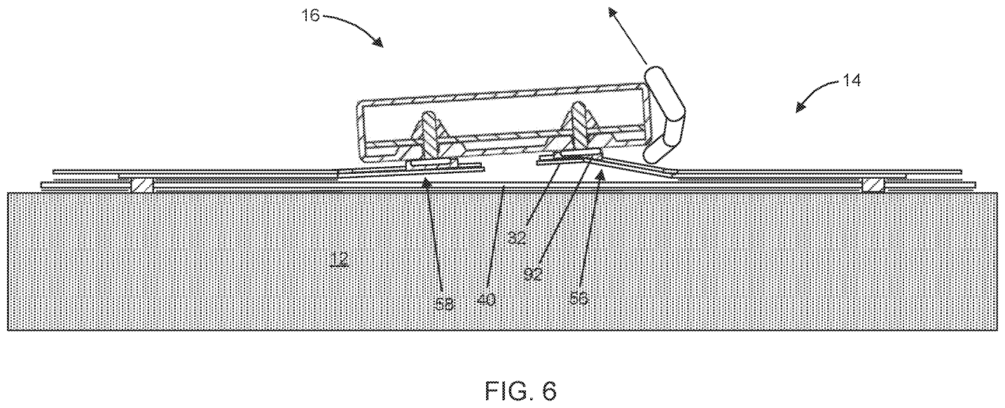

FIG. 6 illustrates demounting the data recording module 16 from the flexible patch 14 by hand and without tools, in accordance with many embodiments. After first demounting the water-resistant cover 18 from the flexible patch 14 by peeling the attachment flange 80 from the upper layer 26, the data recording module 16 can be grasped by hand and rotated relative to the flexible patch 14 to separate the module first electrical contact 68 from the conductive adhesive 92 and the underlying patch first electrical contact 32. During decoupling of the module first electrical contact 68 from the flexible patch 14, the first tab portion 56 is free to move relative to the biocompatible layer 40 due to the lack of adhesive between the first tab portion 56 and biocompatible layer 40, thereby allowing the first tab portion 56 to become angled relative to the bottom surface of the data recording module 16. The resulting tension in the first tab portion 56, coupled with the resulting relative angle between the first tab portion 56 and the bottom surface of the data recording module 16 serves to help separate the first tab portion 56 from the data recording module 16 via peeling. Once the first tab portion 56 is separated from the data recording module 16, the data recording module 16 can be rotated in the opposite direction to separate the second tab portion 58 from the data recording module 16. In addition to rotating the data recording module 16 relative to the flexible patch 14, the data recording module 16 can also be pulled away from the flexible patch 14 to further induce separation of the data recording module 16 from the flexible patch 14. The flexible patch 14 can then be removed from the user 12.

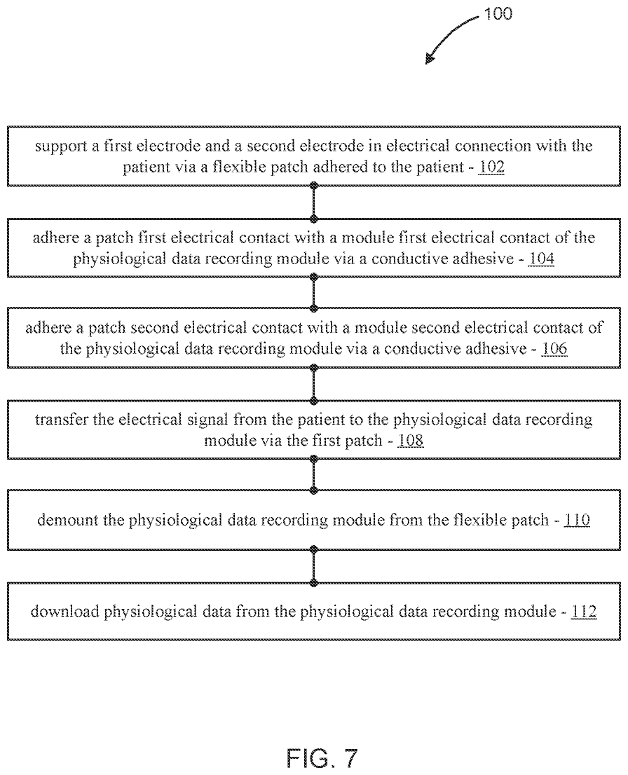

FIG. 7 is a simplified block diagram of acts of a method 100 for transferring an electrical signal from a patient to a physiological data recording module. Any suitable physiological data recording module, such as the data recording module 10 described herein, can be used to practice the method 100. The method 100 includes supporting a first electrode and a second electrode in electrical connection with the patient via a flexible patch adhered to the patient (act 102). A patch first electrical contact is adhered with a module first electrical contact of the physiological data recording module via a conductive adhesive (act 104). A patch second electrical contact is adhered with a module second electrical contact of the physiological data recording module via a conductive adhesive (act 106). The electrical signal is transferred from the patient to the physiological data recording module via the first patch (act 108). The physiological data recording module can be demounted from the flexible patch (act 110). Physiological data (generated and recorded by the physiological data recording module in response to the electrical signal) can be downloaded from the physiological data recording module (act 112).

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1-19. (canceled)

20. An ambulatory monitor comprising:
a flexible patch configured to be attached to a user's skin, the flexible patch including:
a biocompatible layer configured to be coupled with the user's skin;
an upper layer overlying the biocompatible layer;
a first electrode coupled with the upper layer so as to be operatively coupled with the user's skin when the flexible patch is attached to the user's skin;
a patch first electrical contact electrically connected with the first electrode;
a conductive adhesive on the patch first electrical contact;
a second electrode coupled with the upper layer so as to be operatively coupled with the user's skin when the flexible patch is attached to the user's skin;
a patch second electrical contact electrically connected with the second electrode; and
a conductive adhesive on the patch second electrical contact; and
a physiological data recording module including a module first electrical contact and a module second electrical contact, the physiological data recording module being (a) mounted to the flexible patch such that the module first electrical contact contacts the conductive adhesive on the patch first electrical contact and the module second electrical contact contacts the conductive adhesive on the patch second electrical contact and (b) store physiological data of the user generated from an electrical signal from the first and second electrodes.

21. The ambulatory monitor of claim 20, wherein:
the upper layer comprises an upper layer surrounding portion to which each of the patch first and second electrodes is mounted, an upper layer first tab portion to which the patch first electrical contact is mounted, and an upper layer second tab portion to which the patch second electrical contact is mounted;
the upper layer surrounding portion forms an aperture into which each of the upper layer first and second tab portions extend;
the flexible patch comprises an upper layer adhesive layer configured to bond the upper layer surrounding portion to the biocompatible layer; and
at least a portion of each of the upper layer first and second tab portions extends beyond the upper layer adhesive layer so that at least a portion of each of the upper layer first and second tab portions is not bonded to the biocompatible layer.

22. The ambulatory monitor of claim 21, wherein:
the upper layer first tab portion comprises a first end portion, a second end portion, and a central portion disposed between the first end portion and the second end portion;
the upper layer second tab portion comprises a first end portion, a second end portion, and a central portion disposed between the first end portion and the second end portion;
the flexible patch includes an end portion adhesive layer coupled with each of the first and second end portions of each of the upper layer first and second tab portions to bond the data recording module to each of the first and second end portions of each of the upper layer first and second tab portions;
the patch first electrical contact is mounted to the central portion of the upper layer first tab portion; and
the patch second electrical contact is mounted to the central portion of the upper layer second tab portion.

23. The ambulatory monitor of claim 22, wherein the end portion adhesive layer comprises a non-conductive adhesive.

24. The ambulatory monitor of claim 20, wherein the physiological data recording module comprises a housing having a lower surface beyond which each of the module first and second electrical contacts protrude.

25. The ambulatory monitor of claim 20, further comprising a water-resistant cover configured to be bonded to the flexible patch, the water-resistant cover being configured to cover the physiological data recording module when bonded to the flexible patch to protect the physiological data recording module from moisture ingression.

26. The ambulatory monitor of claim 20, comprising:
a first electrical trace formed on the upper layer and electrically connecting the first electrode with the patch first electrical contact; and
a second electrical trace formed on the upper layer and electrically connecting the second electrode with the patch second electrical contact.

27. The ambulatory monitor of claim 20, wherein the user can demount the physiological data recording module from the flexible patch by hand.

28. The ambulatory monitor of claim 20, wherein:
each of the first and second electrodes is formed on the upper layer;
the biocompatible layer has a first electrode aperture aligned with the first electrode and a second electrode aperture aligned with the second electrode;
the flexible patch comprises a conductive material interfaced with the first electrode and extending through the first electrode aperture for electrically coupling the first electrode to the user's skin; and
the flexible patch comprises a conductive material interfaced with the second electrode and extending through the second electrode aperture for electrically coupling the second electrode to the user's skin.

29. The ambulatory monitor of claim 20, wherein:
the flexible patch further includes:
a third electrode coupled with the upper layer so as to be operatively coupled with the user's skin when the flexible patch is attached to the user's skin;
a patch third electrical contact electrically connected with the third electrode; and
a conductive adhesive interfaced with the patch third electrical contact; and
the physiological data recording module includes a module third electrical contact, the physiological data recording module being configured to:
be mounted to the flexible patch so as to electrically couple the module third electrical contact with the patch third electrical contact; and
store physiological data of the user generated via the first, second, and third electrodes.
